(19) United States
(12) Patent Application Publication
(10) Pub. No.: US 2004/0022784 A1
(43) Pub. Date: Feb. 5, 2004

(54) Oligosaccharide supports for e.g. removal of antibodies from blood

(76) Inventor: Kurt Nilsson, Lund (SE)

Correspondence Address: Smith Gambrell & Russell, Suite 800, 1850 M Street NW, Washington, DC, 20036, US

(21) Appl. No.: 09/958,272

(22) PCT Filed: Feb. 7, 2001
(86) PCT No.: PCT/SE01/00241

(30) Foreign Application Priority Data
Feb. 8, 2000 (SE) 0000430-9
May 16, 2000 (SE) 0001833-3
Jun. 28, 2000 (SE) 0002462-0
Nov. 24, 2000 (SE) 0004343-8

(51) Int. Cl. A61K 39/00; C08B 37/00; C02F 1/44

(52) U.S. Cl. 424/140.1; 210/645; 536/123

(57) ABSTRACT

Material characterized by that the material contains at least one biologically active saccharide which is covalently bound via at least one spacer to a cross-linked matrix and that the material is autoclaved.

A material containing so-called matrix to which saccharide has been bound via a spacer is described below.

The active part of the material according to the invention, contains at least one Saccharide part which has been bound via a spacer to a Matrix according to:

Saccharide-spacer-Matrix.

The Matrix consists in general of a polymer, plastic, or a polysaccharide, and can bind a large number of Saccharide-spacer units.

Saccharide symbolizes saccharide which has a biological or other affinity to another molecule, protein virus or cell. Saccharide can consist of a glycoprotcin, a neoglycoprotein, a glycopeptide or a glycosylated amino acid, a glycolipid, or a part, a fragment or a modified variant thereof, or another biologically active di- or tisaccharide or higher oligosaccharide substance.

A few non-limiting examples of biologically active saccharides, spacer and Matrix which can be used according to the invention, are given below.

The active part of the Material consists, as a non-limiting example, of for example either.

1. Blood group A—O(CH₂)ₙPhNH—CO—(CH₂)ₘNH—CH₂—CH(OH)—CH₂—O-Matrix or:

2. Blood group B—O(CH₂)ₙPhNH—CO—(CH₂)ₘNH—CH₂—CH(OH)—CH₂—O-Matix

where Matrix denotes e.g. a plastic or a polysaccharide, for example cross-inked agarose, specifically of the type Sepharose^{R} Fast Flow, where —O(CH₂)ₙPhNH—CO—(CH₂)ₘNH—CH₂—CH(OH)—CH₂— is spacer, according to the invention, to separate the Saccharide, in above examples blood group determinant A- and B-, respectively, from the Matrix,

where n and m, respectively, is an integer, n is for example one of 0, 1, 2, 3 or 4, and m is for example 1, 2, 3, 4, 5, 6 or 7, and where the linkage between —O— and Matrix is formed between —O— and for example a carbon atom in the Matrix.

Saccharide-spacer, for example Blood group A—O(CH₂)ₘPhNH—CO—(CH₂)ₘNH— and Blood group B—O(CH₂)ₙPhNH—CO—(CH₂)ₘNH—, respectively, is below called (the) ligand. The Matrix has a lard number of bound molecules of ligand. Examples of bound amount of ligand is 0.01, 0.1, 1, 2, 3, 4, 5, 6, 7, 8, 9, 10, 11, 12, 13, 14, 15, 16, 17, 18, 19, 20 mmole per liter of Matrix, or an amount of mmole which is between two of the above given values per liter of Matrix. Per liter Matrix here means the volume occupied by the ready-to-use Matrix product.

A combination of two or more different saccharides can be used according to the invention, for example as non-limiting example, a combination of Blood group A—O(CH₂)ₙPhNH—CO—(CH₂)ₘNH—, and Blood group B—O(CH₂)ₙPhNH—CO—(CH₂)ₘNH—, where both ligands in this example are bound to Matrix.

A non-limiting example of a preferred variant of product 1 above is:

1.a GalNAcα1-3(Fucα1-2)Galβ-O(CH₂)₂PhNH—CO—(CH₂)₅NH—CH₂—C(OH)—CH₂—O-Matrix

This type of product can be produced by reaction betvce for example GalNAcα1-3(Fucα1-2)Galβ-O(CH₂)₂PbNH₂ and for example NHS-activated Sepharose^{R} 4FF, where the latter is commercially available, or cross-linked agarose or other Matrix with corresponding properties. The reaction conditions are chosen by the expert and does not limit the scope of the invention. Other examples are for example product containing a-in the same manner bound, higher oligosaccharide than the A-trsaccharide in the above example, which contains the A-determinant terminally, for example A-determinant of type 1, 2, 3 or 4. The trisaccharide derivative GalNAcα1-3(Fucα1-2)Galβ-O(CH₂)₂PbNH₂ and other saccharide derivatives mentioned in this application can be produced with different chemical and/or biochemical methods and this do not limit the scope of the invention.

Further examples of product 1 is Product where a combination of 1.a. and one or more of mentioned blood group A variants, are bound via the same type of spacer as shown above to Matrix, or via a different type of spacer.

A non-limiting examples of a preferred variant of product 2 above is:

2.b. Galα1-3(Fcaα1-2)Galβ-O(CH₂)₂PhNH—C—(CH₂)₅NH—CH₂—CH(OH)CH₂—O-Matrix

This type of product can be produced by reaction between for example Galα1-3(Fucα1-2)Galβ-O(CH₂)₂PbNH₂ and for example NHS-activated SepharoseR 4FF, where the latter is commercially available. The reaction conditions are chosen by the expert and does not limit the scope of the invention. Other examples are for example product containing a in the same manner bound higher oligosaccharide, which contains the B-determinant terminally, for example B-determinant of type 1, 2, 3 or 4. Further examples of product 2 arc Material where a combination of 2.b. and one or more of mentioned blood group B variants, are bound via the same type of spacer as above to Matrix, or via a different type of spacer.

Instead of the —O(CH₂)₂PbNH— group in the formulas above, another suitable Spacer or part of Spacer can be used, as for example —O(CH₂)ₙNH— or for exanple N(Ac)—CH₂)ₙNH— (Ac=Acetyl group; n is an integer, for example 1, 2, 3, 4, 5, 6, or 7 or higher), or another aliphatic compound, or another aromatic compound.

The saccharide, for example the blood group A- or B-determinant, can also be bound, directly or indirectly, to an oligomeric substance acting as Spacer, or part of Spacer, as for example a mono-, di-, or higher oligosaccharide or polysaccharide, peptide, for example a peptide consisting of amide bound glycine and glutamic acid residues, for example Gly-(Glu-Gly)n-Glu, where n is an integer between for example 1 and 20. In this manner the Saccharide-spacer consists of 1, 2, 3, 4, 5, 6, 7, 8, 9, or 10 or more saceharide units bound to each oligomeric substance or peptide.

The linkage between the saccharide and the peptide can for example be formed via O-glyrosidically bound —O(CH₂)₂PhNH— group (see for example formulas 2.a and 2.b. above), or via for example O-glycosidically bound —O(CH₂)ₙNH—, (where n is an integer for example 1,2,3,4,5,6,7 or higher), where NH— is bound via an amide linkage (NH-CO) to the carboxyl group on the side-chain of the Gluresidues in the peptide. —O in —O(CH₂)₂PhNH— and in —O(CH₂)ₙNH—, respectively, is then bound glycosidically to the Saccharide.

The peptide can first have been coupled to Matrix, for example NHS-activated Sepharose^{R} 4FF via the c-amino-group on the peptide, and thereafter can the saccharide be bound to the peptide via —O(CH₂)₂PhNH—, or for example —O(CH₂)ₙNH—, to the carboxyl group on the Glu-residues in the peptide. This linkage between saccharide and Glu-residues can be achieved by for example carbodiimnide-(for example EDC-) mediated coupling, or by for example succinimide-mediated coupling. The saccharide-spacer can herewith be added to the reaction mixture in for example a desired molar excess in relation to the amount of moles of peptide, e.g. in a molar excess of 2, 3, 4, 5, 6, 7, 8, 9 or 10 times excess or more. These and other reaction conditions are chosen by the expert and do not limit the scope of the invention. Non-limiting examples of in this manner bound amount of Saccharide is 0.01, 0.1, 1, 2, 3, 4, 5, 6, 7, 8, 9, 10, 11, 12, 13, 14, 15, 16, 17, 18, 19, 20 mmole of ligand per liter of Matrix. Per liter of Matrix means here the volume occupied by the ready to use Matrix product.

Another example of peptide is as above, but containing at least one Lysine residue, where the ε-amino group in the Lysine residue of the peptide is used for covalent coupling to Matrix, for example NHS-activated Sepharose^{R} 4FF, with subsequent coupling of, for example, Saccharide-O(CH₂)₂PhNH—, or of for example Saccharide-O(CH₂)ₙNH—, to coupled peptide according to what has been describe above. Other linkages can be used according to the invention.

An advantage of using oligomeric ligands, as above mentioned examples of saccharide-peptide ligands, is that a stronger binding oft-en can be achieved to that which is desired to separated by the product, for example of antibodies towards blood group determinant or of other proteins, viruses or cells, and that therewith a more efficient product can be obtained as compared with non-oligomeric ligand.

As a non-limiting example of one preferred variant of Material according to the invention and as a non-litmitng example of its production may be mentioned the coupling of ca 3, 1.5 and 1 gmol, respectively, of the peptide

Ac-Lys-(e-amino)-Gly-Glu-Gly-Glu-Gly-Glu-Gly-Glu-Gly-Glu-Gly-Glu-Gly-amide via its amino group, to 2 ml of NHS-activated Sepharose 4 FF, from Pharmacia-Biotech, at pH 7.5 (0.2 M sodium fosfate buffer+0.5 M NaCl) under 4 h at room temperature followed by 0.3 M Tris-HCl, pH 8, at room temp., over night. The gel is washed with Tris-buffer and 0.1. M MES, pH 4.7, which gave Peptide-Sepharose 4FF. Sawcharide-spacer, as for example Galα1-3Galα-OPhNH₂, Ca 25 mikromole, dissolved in 0.1 M MES-buffert, pH 4.7, was added to a solution of 48 mg E-DC ((1-Ethyl-3-(3Dimethylatninopropyl)-Carbodiimide)), to which was added 2 mJ of Pepdde-Sepharose 4FF, the mixture was incubated under 4 h at room temperature, after which it was washed with Tris-HCl, pH 8, 0.1 M acetate- and 0.1 M sodium fosfate buffer, respectively. The gels were tested for binding of antibodies, in the example of anti-Galα1-3Gal antibodies and showed a better binding of IgM antibodies as compared with the same amount of Sackarid-spacer-Sepharose 4FF, obtained by coupling of the corresponding amount of Galα1-3Galα-OPhNH—, but without peptide, directly to NHS-activated Sepharose 4FF. Other saccharide derivatives than Galα1-3Galα-OPhNH₂ can be used according to the invention, such as for example GaINAcα1-3(Fucα1-2)Galβ-O(CH₂)₂PhNH₂ or Galα1-3(Fucα1-2)Galβ-O(CH₂)₂PhNH₂.

The saccharide-peptide conjugate can for example first be prepared by using for example the, ε-BOC-derivative (BOC=tert-butyloxycarbonyl group, situated on the ε-amino group of the Lysine residue) of for example the peptide in the above mentioned example. The Saccharide-spacer-peptide konjugat is then first formed by for example EDC-mediated reaction between amino groups on the saccharide-spacer and the carboxyl groups on the peptide, the resulting conjugate can be purified by for example Sephadex chromatography, the BOC group can be eliminated by for example trifluoroacetic acid reaction according to standard conditions for peptide chemistry, and the conjugate can be coupled for example in the same manner as describe above via the e-amino group of the lysine residue to the Matrix, for example NHS-activated Sepharose 4 FF.

As another non-limiting example of peptide can be mentioned peptide consisting of amide bound Gly and Lys units, for examplc Gly-(Lys-Gly)n-Gly, where n is an integer between for example 1 and 20. In this case can for example the peptide be bound to the Saccharide via amino groups on the peptide, a N-glycosidic linkage is formed between the reducing end on the Saccharide and the F-amino group on the Lysine residue(s), and the Saccharide-peptide can be coupled to the Matrix via for example either the remaining amino group(s) on the peptide to for example NHS-activated Sepharose as describe above, or via for example the terminal COO-group on the peptide and amino groups on amino group containing Matrix, for example aminohexyl-Sepharose (by for example carbodiimide or succinimide coupling according to examples given above). The N-glycosidic linkage can be stabilised by acetylation under standard conditions, for example before coupling to the Matrix, e.g. NHS-activated Sepharose 4 FF. In the same manner as for the Gly-Glu-peptide above can also an aliphatic or aromatic spacer be used to bind the Saccharide to the Lysine residues of the peptide, but in this case is, for example, glycosidically bound groups of the type —O(CH₂)₂PhCOO—, or for example O(CH₂)ₙCOO—, used for carbodiimide- or succinimide-mediated coupling between Saccharide and Lysine residues in the peptide.

The coupling to the peptide can also be carried out by first coupling the Saccharide part to the amino acid and thereafter form the peptide linkages.

Further examples of Ligand according to the invention, is to use a protein or a polysaccharide as Spacer, or part of Spacer, between Saccharide and Matrix. Here for example a protein, such as serum album or a polysaccharide, such as dextran, is used. The Saccharide can then first be coupled to the protein, or to the polysaccharide, which then is coupled to the Matrix. The same type of chemistry as exemplified above can, as non-limiting examples, be used to achieve the linkages between Saccharide, protein, or polysaccharide, and Matrix. This do not limit the scope of the invention, and the conditions are chosen by the expert.

To use a peptide, protein or polysaccharide according to what have exemplified above, can in some cases be an advantage to increase the ability of the Material to bind proteins, and thereby increase the efficiency of the product according to the invention.

As another example of Matrix can be mentioned the filters which are used for plasma separation. These can be chemically modified with standard technique and be used for coupling of oligomeric ligand or of non-oligomeric ligand mentioned in this description. In this manner is achieved a product which can be used for specific removal of proteins in connection with bloodplasma separation, for example antibodies directed towards Galα1-3Gal and other so called xeno antigens in connection with xenotrnsplantation.

In a variant of the invention, the product in addition contains a Tris structure according to the following non-limiting example:

(HOCH₂)₃C—NH—CO—(CH₂)₅NH—CH₂—CH(OH)—CH₂—O-Matrix

where (HOCH₂)₃C—NH is a so called Tris-group. This product can be made by reaction between Tris-HCl and for example NHS-activated Sepharose 4 FF in which case Matrix above is Scpharose 4 FF.

In the production of the product according to the invention can for example be uscd commercially available activated Matrix, for example so called NHSactivated Sepharose^{R} 4 Fast Flow (NHS— is an abbreviation of N-hydroxysuccinimide; this variant of agarose is relatively strongly cross-linked, commercially available), which is present in the form of practically spherical particles. The particle size is chosen in, for example, the interval 45-165 μm. This activated Matrix can be used for covalent binding of for example, Blood group A-O(CH₂)ₙPhNH₂—, to give product 1.a. above, and of Blood group B—(CH₂)ₙPhN₂, which give product 2.b. above, respectively, at, as non-limiting and typical example, pH 7.5 or pH 8.0, in buffer, for example 0.1 M sodium phosphate as non-limiting example, under for example 1, or 2 hours or for 20 hours, and in the example at room temperature. The material is washed for example on a glass filter or under other conditions, for example sterile conditions, with for example buffer and is subsequently treated with for example Tris-HCl buffer to react any remaining reactive groups. The expert chooses the conditions for the reactions and this do not limit the scope of the invention. See also the above given example in connection with the preparation of Galα1-3Gal-Peptide-Sepharose 4FF.

In the production of the product according to the invention can, as another example, be used so-called epoxy-activated Sepharose^{R} 4 Fast Flow, to which is covalently bound, for example Blood group A-O(CH₂)ₙPhNH—CO—(CH₂)ₘNH—, or to which is covalently bound Blood group B—O(CH₂)ₙPhNH—CO—(CH₂)ₘNH—,

where n och m are specified above as are Blood group A and Blood group B, respectively.

As has been mentioned above, a combination of bigands can also be covalcntly bound the Matrix.

The products can be used, for example, for extra-corporal removal of blood group A- and blood group B-antibodies, respectively, e.g. for purification of blood, or for example, before a transplantation, for example oer the blood group barrier. The product can be used in geeral for different types of transplantation as a part of the treatment of the recipient before and during, and eventually after the transplantation This to be able to solve the problem of blood group incompatibility between donor and recipient. The product can for this purpose be filled into a column housing with in- and outlet for passage through the column of for example blood plasma, or whole blood, from the patient who shall be transplanted or who is undergoing a transplantation procedure. The use of the product is therefore not restricted to for example, blood group incompatible transplantation, but can also be used, for example, for blood group compatible transplantation, to minimize problems in connection with donor and recipient of the same blood group, but of different blood group subgroups, for example A1, A2 etc.

Other non-limitng examples of Saccharide according to the specific examples 1 och 2 above, are structures where the saccharide part consists of Galα1-3Galα-, Galα1-3Galβ-, Galα1-3Galβ1-4Glcβ-, Galα1-3Galβ1-4GlcNAcβ-, Galα1-3Galβ1-4GlcNAcβ1-3Galβ1-4Glcβ-, or of oligomejic ligands, such as for example (Galα1-3Galα-)n-, (Galα1-3Galβ-)n-, (Galα1-3Galβ1-4Glcβ-,n-, (Galα1-3Gaβ1-4GlcNAcβ-)n-, (Galα1-3Galβ1-4GlcNAcβ1-3Galβ1-4Glcβ-)n-, or (Galα1-3Galα-spacer)n-, (Galα1-3Galβ-spacer)n-, (Galα1-3Gal1-4Glcβ-spacer)n-, (Gal≢1-3Galβ1-4GlcNAβ-spacer)n-, (Galα1-3Galβ1-4GlcNAcβ1-3Galβ1-4Glcβ-spacer)n-,

where n is an integer larger than 1. As mentioned above for other saccharides, different spacers can be used in connection with above mtentioned saccharides. Non-limiting examples of spacer have been given above, for example of non-oligomeric, oligomenric, and of polymeric type, respectively. These structures can be of interest to be used in for example a colemn or in a plasmafilter, for example before and after xenotransplantation to reduce so called xeno-antibodies from the patient's blood (whole blood column) or plasma.

Other carbohydrate structures active towards other antibodies, for example antibodies against cancer-antigens, for example prostate-, breast-, intestine-, or skin cancer, can be used to form Product according to the invention. This can be of interest for example to isolate antibodies from blood or plasma against said antigen and after elution from the Product, be used for treatment of said cancer diseases, or to produce reagents, or for example, to remove an excess of antibody derivatives from blood or plasma in immunotherapy of cancer. Other carbohydrate structures specific for e.g. toxins, virus and/or bacteria, can also be used to form Product according to the invention. These Products can be used to, for example, purify or eliminate virus and/or bacteria from e.g. whole blood or plasma or from other materials, for example food products or from water.

A combination of two or more different ligands (Saccharide-spacer) bound to Matrix, can be used in the Product according to the invention. The saccharides can then be different, and/or the spacer can be different.

The product according to the invention, allows for example a combination of high flow rate (for example in the interval 20-60 ml/min), minimal drop in pressure over the column, and a good binding capacity also of molecularly larger proteins, for example antibodies, such as ISO and IgM. As a non-limiting example can-be mentioned single passage of more than one liter blood group B plasma with a flow rate of ca 40 ml/minute through a column with ca 3 mikromol of blood group A trisaccharide per ml Sepharose 4 FF, and with a total product volume of 62 ml and an average particle size of 90 μm, practically climinated all antibodies reactive against blood group A. Similar result was obtained with blood group B product. The products were built according to 1.a. and 2.b. above from A- and B-trisaccharide-spacer and NHS-activated Sepharose^{R} 4 Fast Flow.

One or more columns can be used. The column volume is chosen for the purpose and can be for example of a size of, for example, 10 ml, 20 ml, 40 ml, 60 ml up to a size of for example 500 ml or higher depending on which volumes are desired to process. The column volume can be for example of a value between the given values. Different types of column houses of different dimensions can be used. The product according to the invention functions, as non-limiting example, in the type of column housing with the dimensions used for the product ImmunoSorba^{R}, (which has protein A as ligand bound to Matrix), which has an inner volume between the porous membranes of ca 62 ml (that is allows filling of 62 ml Material according to the invention).

When using Products according to the invention for treatment of plasma, can in general be used membranes which have a lower porosity and Matrix particles which have lower particle size as compared with the case when the Product is applied for treatment of whole blood. Thus, for example, in the case of treatment of plasma, membrane with porosity of for example 30 micrometer, or membrane with a porosity in the interval 20 till 40 micrometer, and particle size of Matrix of for example 90 micrometer, or Matrix of for example particle size in the interval 40-200 micrometer, can be used. When using Products according to the invention for treatment of whole blood, membrane with porosity of for example 30 micrometer or 70 micrometer, or membrane with a porosity in the interval 20 to 100 micrometer, can be used, and the particle size of the Matrix can be for example 150 micrometer, or the Matrix particle size can be for example in the interval 100-250 micrometer. The porosity is chosen by the expert and does not limit the scope of the invention.

The filling of the Product according to the invention in the column can be done using different principal methods. According to the invention, the Product can for example either be autoclaved first and thereafter be filled aseptically in the column or the Product can, as an example of another preferred form of the invention, first be filled in the column and thereafter is the column with the Product autoclaved.

Non-limiting example of autoclaving is treatment in an autoclave of for example counter-pressure type, which involves treatment under at least 20 minutes at 121° C. or higher and with for example water steam. Other conditions can be chosen by the expert from what is suitable, e.g. sterility and stability of the product. As an example can be mentioned that Saccharide-spacer-Matrix according to examples 1.a. and 2.b., obtained via coupling of the respective ligand to NHS-activated Sepharose 4FF, exhibits the same properties after autoclaving as before autoclaving concerning tested parameters such as antibody binding properties and other properties.

The column completely, or partially, filled with Material according to the invention, can for example be constructed in materials which allows for autoclaving (biocompatible plastic materials which can be autoclaved are commercially available, e.g. column housing and locks are made of special polycarbonate, tubings of PVC or silicon material and rings of silicon) and/or for example to allow aseptic packing of Material according to the invention. Column house exists on the market for extracorporal blood treatment, e.g. Immunosorba. This allows for aseptic filling, but nor for autoclaving.

Non-limiting example of autoclavable column house is a column house built from autoclavable materials. Otherwise can the same principal design as on the market available column houses be used with two locks, both equipped with for example identical threads which are screwed, with help of the threads, outside and at both endings of a cylinder(house), which is equipped with matching threads at the two endings of the cylinder(house). Between each lock and cylinder is, before screwing together the locks and the cylinder, placed a porous membrane (that is two membranes and rings for each column), which allows for passage of plasma or whole blood but not for passage of the Material according to invention. Each membrane is mounted between the lock and the cylinder with for example a silicon ring with a fitting grove of ca the same or the same diameter as the cylinder. Every silicon ring has for example a grove which allows for fitting the circular membrane in the grove in the silicon ring. The membrane is mounted in the silicon ring and is placed between the lock and the ending of the cylinder, after which the lock is screwed on the cylinder as described above. The silicon ring with the membrane is therewith enclosed between the lock and the cylinder ending. The same procedure is carried out for the other ending of the cylinder. Each lock has a centrally placed hole with an elevation which allows for connecting a bio-compatible and autoclavable set of tubings equipped with connections of e.g. the Luer type for connection of other equipment used in extracorporal treatment.

Instead of above mentioned design where the locks and the cylinder are connected with threads, a clip mechanism can, for example, instead be used, where for example the locks are equipped with. one or more clips (for example in each one of the locks there are 2, 3, 4, 5, 6, 7, 8, 9, 10, 11, 12, 13, 14, or more separate clips which are situated beside each other with a defined interspace), and the cylinder has on its outer side one or more protruding edge(s) placed at a distance, e.g. of 2, 3, 4, 5, or 6 mm from the top and from the bottom, respectively, of the cylinder. The clips can be protruding down from the locks and can for example be homogeneous, or each one be equipped with a cavity to allow for a larger flexibility without being broken. In this manner can the silicon ring with the membrane according to the above be placed between the lock and the respective cylinder ending, and the lock is thereafter pressed on the cylinder, whereupon the clips are pressed under de protruding edges on the cylinder and stays there, and the silicon ring with the porous membrane is consequently sealed between the lock and the cylinder.

In order to fill the so mounted column houses with Material according to the invention, the cylinder part can be equipped with a circular opening with a protruding part, which has threads, on the outer side of the cylinder to allow connection of a tubing used for filling of the material. After filling of the material into the column housing, a biocompatible plug with threads which matches the threads of the protruding part on the cylinder, is mounted. In the center of the plug is a protruding tap which fits into the circular hole of the cylinder and which has a length which corresponds to the height of the protruding part In this manner an (almost) flat surface is achieved inside the cylinder at the circular opening.

For the autoclaving of a column filled with Material according to the invention and connected with a set of tunings, can an outer ring of e.g. PVC material, and with an inner diameter which corresponds with the outer diameter of threads of each lock, be mounted around (the threads of) each lock before the autoclaving. This can be done in order to minimize any deformation of the threads under the autoclaving process. This principle has been successfully used under autoclaving of column filled with Material according to the above.

All mentioned components of the column house in a preferred example according to the invention with autoclavable column house, are autoclavable and biocompatible. Lock membrane, cylinder, plug, and tubing with luer coupling can be made of biocompatible plastic material.

Column house completely or partially filled with Material according to the invention and equipped with above mentioned closed tubing set and plug can be autoclaved. This facilitates according to the invention the achievement of sterility of the products according to the invention. With earlier methods sterile (aseptic) filling have been caned out, which are difficult to achieve.

1. Material characterized by that the material contains at least one biologically active saccharide which is covalently bound via a spacer to a cross-linked matrix.

2. Material according to claim 1, which has been treated by autoclaving that is autoclaved Material.

3. Material according to claim 1, completely or partially filled in column consisting of biocompatible material.

4. Column according to claim 3, which has been treated by autoclaving, that is autoclaved Column.

5. Use of any of products mentioned in claims 1 to 4, for treatment of blood, for treatment of blood plasma, for isolation or for elimination of a protein, virus or cell.
